(19) United States
(12) Patent Application Publication
(10) Pub. No.: US 2023/0048812 A1
(43) Pub. Date: Feb. 16, 2023

(54) Cosmetic composition comprising extracts of pomegranate and peony

(71) Applicant: L V M H RECHERCHE, SAINT JEAN DE BRAYE (FR)

(72) Inventors: Jocelyne FRANCHI, SAINT JEAN DE BRAYE (FR); Virginie LEPLANQUAIS, SAINT JEAN DE BRAYE (FR); Emmanuelle LEBLANC, SAINT JEAN DE BRAYE (FR)

(73) Assignee: L V M H RECHERCHE, SAINT JEAN DE BRAYE (FR)

(21) Appl. No.: 17/782,354

(22) PCT Filed: Dec. 11, 2020
(86) PCT No.: PCT/FR2020/052384

§ 371 Date: Jun. 3, 2022

(30) Foreign Application Priority Data
Dec. 12, 2019 (FR) FR1914248

(51) Int. Cl. A61K 8/9789; A61K 8/92; A61Q 19/00

(52) U.S. Cl. CPC A61K 8/9789; A61K 8/922; A61Q 19/00; A61K 2800/591

(57) ABSTRACT

The present invention relates to a cosmetic composition comprising in a physiologically acceptable medium an extract of pomegranate and an extract of peony.

### FIELD OF THE INVENTION

The present invention relates to the field of cosmetics. It especially relates to a particular association of an oily extract of pomegranate and oily extract of peony intended to improve the comfort, emollience and hydration of the surface of keratin material, and to prevent and/or reduce loss of volume and firmness of the skin and/or lips, and in particular of the lips.

### PRIOR ART

The skin is the first protective barrier of the body against the environment. The skin and lips are therefore daily subjected to factors of external or internal origin likely to weaken the skin barrier, promote dehydration of the skin and lips and deteriorate the aesthetic appearance thereof.

Facial skin, and even more so the lips, are a part of the body most particularly sensitive to water loss. Good hydration of the skin and lips and is essential for comfort which especially impacts tactile sensitivity (Effect of hydration on the tactile and thermal sensitivity of the tip, S. Guest, Physiology Et Behavior 123 (2014), 127-135).

There is therefore a constant need to find novel agents capable of nourishing and hydrating the skin and/or lips, restoring the skin barrier and thereby preventing and/or reducing the deleterious effects to which our skin is daily exposed.

Unexpectedly, the Applicant has evidenced the in vitro effects of an association of extracts combining a pomegranate extract and peony extract, in particular an oily extract of pomegranate flowers and oily extract of peony flowers. This association of plant extracts particularly allows modulated expression of some genes having a function related to the hydration, firmness and reinforcement of the skin barrier. It is therefore envisaged to use this association of extracts as cosmetic active ingredient to promote and/or stimulate the hydration and/or nourishment of keratin material, preferably the skin and/or lips, and/or to prevent and/or reduce loss of volume, toss of firmness, loss of elasticity, decline in skin thickness and/or deterioration of the barrier function of the skin and/or lips.

### SUMMARY OF THE INVENTION

The first subject of the invention concerns a cosmetic composition comprising, in a physiologically acceptable medium, an extract of pomegranate and an extract of peony, preferably an extract of pomegranate flowers and an extract of peony flowers.

The invention further concerns a cosmetic method for the care and/or makeup of keratin material, in particular the skin and/or lips, comprising the application to said keratin material of at least one layer of a cosmetic composition of the invention.

By «keratin material» in the invention it is meant the skin and/or its appendages and the lips. In particular it concerns the skin of the face, neck and/or body, and of the lips.

The cosmetic method of the invention is particularly intended to promote and/or stimulate the hydration and/or nourishment of keratin material, preferably the skin and/or lips, and/or to prevent and/or reduce toss of volume, toss of firmness, toss of elasticity, decline in skin thickness and/or deterioration of the barrier function of the skin and/or lips.

The present invention further concerns the cosmetic use of an association of plant extracts of the invention, in particular the association of at least one extract of pomegranate flowers in jojoba oil, and an extract of peony flowers in sweet almond oil, as association of active ingredients to promote and/or stimulate the hydration and/or nourishment of keratin material, preferably the skin and/or lips, and/or to prevent and/or reduce loss volume, toss of firmness, loss of elasticity, decline in thickness of the skin and/or lips and deterioration of the barrier function of the skin and/or lips.

### DESCRIPTION OF THE FIGURES

FIG. 1 is a graph illustrating the expression of Aquaporin 3 (AQP3) under control conditions or in the presence of extracts of the invention (staining intensity/cell).

FIG. 2 is a graph illustrating the expression of Nidogen under control conditions or in the presence of extracts of the invention (Staining intensity/cell).

FIG. 3 is a graph illustrating the expression of Procollagen 1 under control conditions or in the presence of extracts of the invention (Staining intensity/cell).

FIG. 4 is a graph illustrating the expression of fibronectin under control conditions or in the presence of extracts of the invention (Staining intensity/cell).

### DETAILED DESCRIPTION OF THE INVENTION

### Plant Extracts of the Invention

In one aspect, the present invention relates to an association of plant extracts, in particular the association of an extract of pomegranate and an extract of peony.

By plant «extract» it is generally meant an isolated substance, obtained by extraction from a plant raw material and which does not pre-exist as such in nature.

The plant extracts according to the invention are used in cosmetic compositions, in the form of a solution or concentrate, otherwise called «product containing said extract», the solvent of the concentrate possibly being the extraction solvent and/or an additional solvent. The extracts illustrated in the examples of compositions are therefore in the form of a solution. The terms products or products containing each of the extracts will also be used, some of said products being commercial products.

It is known to persons skilled in the art that plants, or the vegetable kingdom in general, can produce numerous substances of cosmetic interest. Depending upon the type of plant or flower used, these substances of interest can be found in different parts or different organs of said plant, in particular in the seeds, roots, bulbs, stems, leaves, petals, flowers, flowering heads, even in the whole plant. For one same plant, or for one same flower, the different extracts and in particular the extracts from seeds, roots, bulbs, stems, leaves, petals, flowers, flowering heads will have different compositions of active molecules and will therefore exhibit complementary and even synergic cosmetic properties.

In the present invention, said association is characterized in that each of the plant extracts is obtained from a part of said plant selected from the group composed of seeds, roots, bulbs, stems, leaves, petals, flowers, flowering heads, whole plant, and mixtures thereof.

In one particular embodiment, each of the plant extracts is obtained from a part of said plant selected from the group composed of petals, flowers, flowering heads and mixtures thereof, preferably the plant extracts of the invention are obtained from the flowers and/or flower petals.

Therefore, according to one preferred embodiment, the invention relates to the association of an extract of pomegranate flowers and an extract of peony flowers.

In one particular and preferred embodiment, each of the plant extracts of the invention is an «oily extract». By «oily extract» in the invention, it is meant an extract comprising non-volatile compounds. Therefore, the plant extracts of the invention differ from an «essential oil» which is composed of volatile compounds. An essential oil is obtained by extraction or distillation of volatile molecules from the plant material (e.g. terpenoids, aromatic molecules).

Numerous methods for extracting plant raw material are known in the prior art and skilled persons can easily implement or, if need be, adapt these known methods to prepare plant extracts of the invention. Nonetheless, the plant extracts of the invention are preferably obtained using a green extraction method.

By «green extraction» it is meant an extraction method allowing energy savings, but also the use of alternative solvents and renewable plant resources, whilst guaranteeing a product/extract that is safe and of quality. Green extraction prioritises 6 main principles (Farid Chemat, Dunod, 2010, Eco-Extraction du vegetal):
Promotion of innovation through selection of varieties and use of renewable plant resources.
Priority given to alternative solvents called green solvents and chiefly those derived from agricultural resources;
Energy savings with the aid of innovative technologies, and promotion of energy recovery.
Production of co-products instead of waste, to integrate bio- and agri-refining.
Reduction of unit operations through technological innovation, and promotion of safe, robust, controlled processes.
Priority given to non-denatured, biodegradable products free of contaminants and especially exhibiting «green extract» values.

As green solvent, advantageous use is made of: water or a vegetable oil.

In one particular embodiment, the invention concerns the association of an oily extract of pomegranate flowers and an oily extract of peony flowers, said oily extracts preferably being obtained by means of the above-described extraction methods.

Pomegranate Extract

The present invention uses a pomegranate extract, preferably a pomegranate extract from the species Punica granatum, a fruit-bearing tree in the family Lythraceae, originating in Western Asia and cultivated in tropical and sub-tropical regions.

In the context of the invention, the pomegranates used to obtain the plant extracts are preferably of Moroccan origin.

In the context of the present invention, the pomegranate plant extract is obtained from part of said plant selected from the group composed of the seeds, roots, bulbs, stems, leaves, petals, flowers, flowering heads, whole plant, and mixtures thereof. In one preferred embodiment said pomegranate extract, Punica granatum, is a flower extract having the INCI name (International Nomenclature Cosmetic Ingredient): Punica granatum flower extract.

In one particular embodiment, said pomegranate extract is obtained via oil extraction with the patented Oleo-eco-extraction process described in particular in European patent EP2413706 (B1). Advantageously, this process allows the extraction of natural, non-volatile compounds contained in pomegranate flowers using a natural fatty substance, in particular a vegetable oil or wax and preferably jojoba wax, without the use of an organic solvent and without chemical conversion.

Therefore, in one particular, preferred embodiment, said pomegranate extract used in the present invention is obtained with an extraction method comprising at least the steps of:
a) mixing and impregnating the solid raw material, preferably pomegranate flowers, in dispersible form with the natural fatty substance, preferably jojoba wax, at a temperature higher than the melting point of said fatty substance and in an atmosphere free or essentially free of oxygen;
b) heating to a temperature of between 80 and 200° C. for a time of 10 min or less and in an atmosphere free or essentially free of oxygen, using microwaves;
c) micro-dispersing the material to be extracted and optionally disrupting the cells of the raw material in said natural fatty substance at a temperature higher than the melting point of the fatty substance and in an atmosphere free or essentially free of oxygen, using microwaves, step c) possibly being performed before, during or after step b).

In this particular embodiment, the pomegranate flowers are previously crushed, preferably crushed at low temperature e.g. at between −20 and −80° C. Crushing at low temperature advantageously allows preservation of the active molecules contained in the plant material.

Therefore, said pomegranate extract used in the invention is preferably an oily extract of pomegranate flowers and further preferably an extract of pomegranate flowers in jojoba wax.

This oily extract of pomegranate has advantageous properties for cosmetic use. Pomegranate flowers are high in polyphenols, particularly in ellagic acid and in anthocyanins: antioxidant molecules. Also, liquid jojoba wax has a composition close to that of cutaneous sebum, being 98% composed of C20:1 esters which have a strong affinity for the lipids of the epidermal intercellular cement.

The use of pomegranate flower extract in jojoba wax is therefore of particular advantage for implementing the cosmetic methods of the invention, since this extract particularly promotes hydration, emollience and takes part in skin restructuring properties (firmness, elasticity).

In one preferred embodiment, the pomegranate extract used in the present invention is the product marketed under the trade name «Plump Oléoactif®» by Oléos. The «Plump Oléoactif®» product is referenced in international INCI nomenclature: Simmondsia Chinensis (Jojoba) Seed Oil (and) Punica Granatum Flower Extract (and) Potyglyceryl-3 Diisostearate.

The pomegranate extract is contained in the cosmetic composition of the invention in an effective amount to obtain the desired effect.

In particular, said pomegranate extract, preferably the product marketed under the trade name «Plump Oléoactif®» is contained in an amount ranging from 0.0001% to 10%, in particular 0.0005% to 5%, preferably 0.001% to 1%, more preferably 0.005% to 0.5%; further preferably from 0.01% to 0.4% even 0.05% to 0.3% by weight of raw material (weight of product) relative to the total weight of the composition.

Peony Extract

The present invention uses a peony extract, preferably an extract of officinal peony from the species Paeonia officinalis, an herbaceous plant originating in southern and central Europe.

In the context of the invention, the peonies used to obtain the plant extracts are preferably of French origin.

In the context of the present invention, the peony plant extract is obtained from a part of said plant selected from the group composed of the seeds, roots, bulbs, stems, leaves, petals, flowers, flowering heads, the whole plant, and mixtures thereof. In one preferred embodiment said peony extract, from Paeonia officinalis, is a flower extract (INCI name: Paeonia officinalis flower extract).

In one particular embodiment, said peony extract is obtained by maceration in sweet almond oil.

In one particular embodiment, said sweet almond oil is obtained with a method comprising the steps of:
a) Cold pressing sweet almonds;
b) Decolourizing the virgin oil on neutral earth; and
c) Steam deodorization of the virgin oil.

After step c) the sweet almond oil obtained is a refined sweet almond oil.

Therefore, in one particular and preferred embodiment, said peony extract used in the present invention is obtained with an extraction method comprising the steps of:
a) Cold pressing sweet almonds;
b) Decolourizing the virgin oil on neutral earth;
c) Steam deodorizing the virgin oil, and
d) Macerating the peony plant material in the refined sweet almond oil obtained at step c).

In one particular embodiment the plant materials, preferably the flowers and more preferably the peony petals, are left to dry prior to maceration in said sweet almond oil.

Therefore, said peony extract used in the invention is preferably an oily extract of peony flowers, and more preferably an extract of peony flowers in sweet almond oil.

Advantageously, the sweet almond oil is referenced in the French Pharmacopeia and can be used in the cosmetic compositions of the invention. In addition, sweet almond oil has advantageous properties for cosmetic use. Traditionally used to soften the skin, sweet almond oil has soothing properties.

Sweet almond oil particularly has a high omega-6 content. These fatty acids take part in the fluidity of skin cell membranes and are therefore essential for maintaining a healthy skin. Sweet almond oil is also high in linoleic acid (C18: 2n-6), known for its restructuring and protective effects, this fatty acid is included in the composition of ceramides and contributes towards strengthening the protective skin barrier. Yet, it is known that the presence of linoleic acid in the skin is largely reduced with age (Hayashi 2003). Sweet almond oil also has a high omega-9 content and in particular of oleic acid (C18:1 n-9). These omegas-9 are known for their nourishing property, and are therefore of particular advantage for use on dry and damaged skin, in particular the lips, where they contribute towards creating a film-forming barrier on the skin surface thereby opposing moisture evaporation. Oleic acid is also less abundant in the skin with age.

The use of an extract of peony flowers in sweet almond oil is therefore of particular advantage for use in the cosmetic methods of the invention, this extract particularly promoting hydration and nourishment of the skin and/or lips and of the skin barrier.

In one preferred embodiment, the peony extract used in the present invention is the product marketed under the trade name «Pivoine de France extrait huileux (amande douce raffinée)» by Greentech. This product is referenced in the international MCI nomenclature under: Prunus Amygdalus dulcis (sweet almond) oil (and) Paeonia officinalis flower extract.

The peony extract is contained in the cosmetic composition of the invention in an effective amount to obtain the desired effect.

In particular, said peony extract, preferably the product marketed under the trade name Pivoine de France extrait huileux (amande douce raffinée) is contained in an amount ranging from 0.0001% to 10%, in particular 0.0005% to 5%, preferably 0.001% to 1%, more preferably 0.005% to 0.5%; further preferably from 0.01% to 0.4% even from 0.05% to 0.3% by weight of raw material (weight of product) relative to the total weight of the composition.

Cosmetic and Galenic Compositions of the Invention

In the present invention, this association of extracts is particularly used to prepare cosmetic compositions.

Therefore, in one aspect, the present invention concerns a cosmetic composition comprising, in a physiologically acceptable medium, a pomegranate extract and peony extract.

By «physiologically acceptable» or «cosmetically acceptable» it is meant that the composition is suitable for topical use, in contact with mammalian skin and/or lips, more particularly human skin and/or lips and preferably the lips.

In one preferred embodiment, said pomegranate extract is an extract of pomegranate flowers, and more preferably an oily extract of pomegranate flowers.

In another preferred embodiment, said peony extract is an extract of peony flowers, and more preferably an oily extract of peony flowers.

In one preferred embodiment, said pomegranate extract and said peony extract are prepared with the above-described extraction methods. Preferably, said oily extract of pomegranate flowers is extracted in jojoba oil. Just as preferably, said oily extract of peony flowers is extracted in sweet almond oil.

In some particular embodiments, said cosmetic composition also comprises shea butter (Butyrospermum parkii).

Shea butter has fully advantageous properties in the cosmetic methods of the invention.

When contained in the cosmetic compositions of the invention, this butter contributes towards nourishing and protecting lip skin, it promotes skin renewal and protects against skin dryness. Shea butter therefore provides the skin or lips with protection against exogenous attack such as severe weather, cold, wind or sun.

Preferably, the shea butter used in the cosmetic compositions of the invention is organic shea butter obtained with a natural process having no environmental impact and free of solvent and preserving agent. Also preferably, the shea butter used in the cosmetic compositions of the invention is a butter enriched with unsaponifiable fraction.

By «unsaponifiable fraction», it is meant the non-glyceride, water-insoluble fraction obtained after saponification. Contained in small proportion in vegetable oils, the unsaponifiable fraction is particularly rich in polyphenols, phytosterols, minerals, vitamins, and other antioxidant enzymes.

In the present invention, the shea butter enriched with unsaponifiable fraction is preferably obtained with the supercritical CO2 technique.

Therefore, in one best preferred embodiment, the present invention concerns a cosmetic composition comprising, in a physiologically acceptable medium:
a. an extract of pomegranate flowers in jojoba oil, preferably an extract of Punica Granatum flowers in jojoba oil;
b. an extract of peony flowers in sweet almond oil, preferably an extract of Paenonia officinalis flowers in sweet almond oil; and
c. optionally, shea butter.

In one preferred embodiment, the composition of the invention comprises:
an oily extract of pomegranate flowers, in particular the extract marketed under the trade name «Plump Oléoactif®» by Oléos and having the INCI name: Simmondsia Chinensis (Jojoba) Seed Oil (and) Punica Granatum Flower Extract (and) Polyglyceryl-3 Diisostearate; and
An oily extract of peony flowers, in particular the extract marketed under the trade name «Pivoine de France extrait huileux (amande douce raffinée)» by Greentech and having the INCI name: Prunus Amygdalus dulcis (sweet almond) oil (and) Paeonia officinalis flower extract.

In the invention, the oily extract of pomegranate and oily extract of peony are used in an effective amount to obtain the desired effect. They are preferably used in association and incorporated in a cosmetic composition adapted for topical application to keratin material, in particular to the skin and/or lips, and preferably the lips.

In one particular embodiment, the oily extract of pomegranate and oily extract of peony are preferably used in association, and are each contained in said cosmetic composition in an amount ranging from 0.0001% to 10%, preferably 0.0005% to 5%, more preferably 0.001% to 1% by weight of raw material relative to the total weight of the composition.

In one particular and preferred embodiment, each of the extracts is contained in the composition in an amount ranging from 0.1% to 1% by weight, more preferably 0.2 to 0.5% by weight relative to the total weight of the composition.

In one preferred embodiment, the composition of the invention comprises:
an oily extract of pomegranate flowers, in particular the extract marketed under the trade name «Plump Oléoactif®» by Oleos having the INCI name: Simmondsia Chinensis (Jojoba) Seed Oil (and) Punica Granatum Flower Extract (and) Polyglyceryl-3 Diisostearate, in an amount ranging from 0.001 to 1%, preferably 0.1 to 1% and more preferably 0.2 to 0.5% by weight relative to the total weight of the composition, and
an oily extract of peony flowers, in particular the extract marketed under the trade name «Pivoine de France extrait huileux (amande douce raffinée)» by Greentech having the INCI name: Prunus Amygdalus dulcis (sweet almond) oil (and) Paeonia officinalis flower extract, in an amount ranging from 0.001 to 1%, preferably 0.1 to 1%, more preferably 0.2 to 0.5% by weight relative to the total weight of the composition.

In one preferred embodiment, the cosmetic composition of the invention is a care and/or makeup composition for keratin material, in particular the skin and/or lips and in particular the lips.

The cosmetic composition of the invention comprises at least one pomegranate extract, at least one peony extract, optionally rhea butter, and additionally one or more cosmetically acceptable excipients among those known to persons skilled in the art, to obtain a composition for topical application.

The invention therefore also concerns a cosmetic composition in the form of a topical composition for the lips and/or skin, in particular in the form of a lip balm, stick, lip varnish, cream, oil-in-water or water-in-oil emulsion or multiple emulsion, a solution, suspension get, milk, lotion or serum.

In one preferred embodiment, said composition is in the form of a lip balm, a stick or lip varnish.

In another preferred embodiment, said composition is in the form of a skin cream or oil.

The composition of the invention may also comprise at least one cosmetic additive selected from the group composed of antioxidant agents, emollient agents, hydrating agents, anti-ageing agents, fragrances, and mixtures thereof.

The cosmetic additives advantageously used in the composition of the invention can be contained in an amount ranging from 0.0001% to 10%, preferably 0.001% to 5% by weight (of raw material) relative to the total weight of the composition.

Depending on the type of composition, it is also possible to select one or more cosmetically acceptable excipients from among polymers, surfactants, rheological agents, fragrances, electrolytes, pH adjusters, preserving agents, colouring agents, pearlescent agents, pigments and mixtures thereof.

Depending on the desired galenic form, persons skilled in the art are able to adapt the type and content of aqueous phase and/or fatty phase.

In one embodiment, the composition of the invention is an anhydrous composition.

By «anhydrous», it is particularly meant that water is preferably not purposefully added to the compositions but may be present in trace form in the different compounds used in the compositions. In particular, the composition of the invention comprises less than 4% by weight of water, preferably less than 3%, more preferably less than 2%, further preferably less than 1%, and still further preferably less than 0.5% by weight of water relative to the total weight of said composition, and is even fully free or water.

In another embodiment, the composition of the invention comprises an aqueous phase.

The aqueous phase, when present, generally represents from 1 to 99% by weight relative to the total weight of said composition. The fatty or oily phase will generally range from 0.2% to 45%, preferably 0.5% to 30%, more preferably 2% to 25% by weight relative to the total weight of said composition.

Aqueous Phase

In some embodiments, the cosmetic composition of the invention comprises an aqueous phase. The aqueous phase of the composition of the invention comprises water and optionally a water-soluble solvent.

By «water-soluble solvent» according to the invention, it is meant a compound that is liquid at ambient temperature and water-miscible (miscibility in water greater than 50% by weight at 25° C. and at atmospheric pressure). Particular mention can be made of:
lower C1-C5 mono-alcohols such as ethanol, isopropanol and mixtures thereof;
C2-C8 glycols such as ethylene glycol, propylene glycol, 1,3-butylene glycol, dipropylene glycol, and mixtures thereof;
C2-C32 polyols such as polyglycerols, polyethylene glycols, and mixtures thereof;
and mixtures thereof.

It may also comprise hydrophilic getting agents, antioxidants, preserving agents and mixtures thereof.

As «hydrophilic gelling agents», particular mention can be made of polymers of acrylic acid, copolymers of acrylic and methacrylic acids, carboxyvinyl polymers, associative thickeners of acrylic or polyurethane type, polysaccharide gelling agents, natural or modified gums, mineral gelling agents, and mixtures thereof.

The content of hydrophilic gelling agent(s) in the cosmetic composition of the invention will generally range from 0.01% to 5%, in particular 0.1% to 4%, preferably from 0.2% to 3% and more preferably from 0.5% to 2% by weight relative to the total weight of said composition.

Fatty or Oily Phase

In some embodiments, the cosmetic composition of the invention comprises a fatty phase (solid fatty substances) or oily phase.

By «oily phase» it is meant an oil or mixture of oils which may or may not be miscible with each other.

By «oil», in the invention it is meant a fatty substance that is not water-soluble and liquid at 25° C. and at atmospheric pressure.

These oils can be volatile or non-volatile, vegetable, mineral or synthetic.

By «volatile oil» in the invention, it is meant an oil of volatile nature as defined by at least one of the criteria given below.

Volatility in the invention can be defined for example by vapour pressure measurable at 25° C. with an empirical method, and having a value of between 2.6 Pa and 40 000 Pa, for example between 5 Pa and 20 000 Pa, between 10 Pa and 8 000 Pa, even between 15 and 150 Pa. Vapour pressure is to be measured according to one of the best adapted methods for the compound of interest, in particular the method recommended in the document «OECD Guidelines for the Testing of Chemicals-Vapour pressure», (n° 104 agreed on 23 Mar. 2006). Alternatively, a volatile oil can be chosen having a boiling point at atmospheric pressure lower than 250° C., preferably tower than 230° C. and more preferably between 150′C and 220° C. Finally, the volatile oil can be defined as an oil having a flash point ranging from 35° C. to 100° C., preferably between 40° C. and 80° C.

By «non-volatile oil» in the invention, it is meant an oil not meeting the criteria of volatility defined above, in particular an oil having a vapour pressure measurable at 25° C. with an empirical method tower than 2.6 Pa, preferably lower than 0.13 Pa, measured in particular with the above-described method.

An oily phase of the invention may comprise oils of natural origin, hydrocarbon oils, silicon-containing oils, and mixtures thereof.

These oily compounds can be volatile or non-volatile, liquid or solid at 30° C., vegetable, mineral or synthetic.

By «oil of natural origin» in the invention, it is meant vegetable oils extracted from plants, roots, leaves or fruits, optionally converted by esterification.

By «hydrocarbon oil» in the invention it is meant an oil compound chiefly containing hydrogen and carbon atoms.

As hydrocarbon, non-volatile oil compounds, particular mention can be made of hydrocarbon oils, hydrocarbon oils of vegetable origin, C10-C40 synthetic ethers, C10-C40 synthetic esters, C12-C26 fatty alcohols, C12-C22 higher fatty acids, and mixtures thereof.

By «silicon-containing oil» in the invention it is meant an oil containing at least one silicon atom and in particular at least one Si—O group.

The fatty or oily phase may also comprise waxes, pasty fatty substances, and mixtures thereof.

By «wax» in the present invention, it is meant to designate a compound solid at 25° C. with reversible change from solid/liquid state and having a melting point higher than 30° C., preferably higher than 45° C.

Particular mention can be made of polyethylene waxes, candelilla wax, beeswax, and mixtures thereof.

A composition of the invention, in particular a composition for the lips, may have a wax content ranging from 5% to 25%, for example 10% to 20% by weight, preferably 12% to 18% by weight relative to the total weight of the composition.

By «pasty fatty compound» or «pasty compound» or «pasty fatty substance», it is designated a non-crystalline fatty compound having a liquid fraction and a solid fraction at a temperature of 25′C.

For example, the pasty compound is selected from the group composed of lanoline and derivatives thereof, fatty acid triglycerides and derivatives thereof, shea butter, cocoa butter, mango oil or butter, and mixtures thereof.

A composition of the invention, in particular a composition for the lips, may have a content of pasty compounds ranging from 5% to 25%, for example 10% to 22% by weight, preferably 15% to 20% by weight, relative to the total weight of the composition.

Cosmetic Methods and Uses

The cosmetic methods and cosmetic uses described below are intended for application to keratin material, in particular healthy keratin material, preferably healthy skin and/or lips and in particular healthy lips (‘healthy’ subjects).

By «healthy keratin material» according to the invention, it is meant keratin material not suffering from disorders relating to a pathological condition (‘non-healthy’ subjects suffering from a pathology). The terms skin and/or lips, or healthy skin and/or lips, will be used indifferently in the remainder of the description.

A further subject of the invention concerns a cosmetic method for the care and/or makeup of healthy keratin material, in particular of the skin and/or lips, comprising the application to said keratin material of at least one layer of a cosmetic composition such as described above.

In the present invention, said cosmetic method is intended to promote and/or stimulate the hydration and/or nourishment of keratin material, preferably the skin and/or lips, and/or to prevent and/or reduce toss of volume, toss of firmness, toss of elasticity, decline in skin thickness and/or deterioration of the barrier function of the skin and/or lips.

Consequently, in particular via its effects on hydration, said cosmetic method of the invention allows an improvement in the feeling of comfort of keratin material, in particular feeling of comfort of the skin and/or lips.

A further subject of the invention is the cosmetic use of an association of plant extracts such as described above, in particular the association of at Least one extract of pomegranate flowers in jojoba oil and an extract of peony flowers in sweet almond oil, as association of active ingredients to promote and/or stimulate the hydration and nourishment of keratin material, preferably the skin and/or lips, and/or to prevent and/or reduce toss of volume, loss of firmness, toss of elasticity, decline in skin thickness and/or deterioration of the barrier function of the skin.

In one particular embodiment, the compositions used in the method of the invention comprise:
an oily extract of pomegranate flowers, in particular the extract marketed under the trade name Plump Oléoactif® by Oleos and having the INCI name: Simmondsia Chinensis (Jojoba) Seed Oil (and) Punica Granatum Flower Extract (and) Polyglyceryl-3 Diisostearate; and
an oily extract of peony flowers, in particular the extract marketed under the trade name «Pivoine de France extrait huileux (amande douce raffinée)» by Greentech and having the INCI name: Prunus Amygdatus dulcis (sweet almond) oil (and) Paeonia officinalis flower extract.

In one particular embodiment, the compositions of the invention are to be applied to healthy subjects having toss of skin firmness and/or elasticity and/or volume, or dry skin.

In another particular embodiment, the compositions of the invention are to be applied to healthy subjects having toss of tip volume, or dry or easily irritated lips.

The present invention is illustrated below in the following nonlimiting examples.

Percentages are expressed by weight of raw material relative to the total weight of the composition, unless otherwise stated.

### Examples

### 1. Materials and Method 1.1. Oily Extracts of Pomegranate and Peony

The extracts used in the examples below are:
Oily extract of pomegranate flowers: «Plump Oléoactif®» by Oleos and having the INCI name: Simmondsia Chinensis (Jojoba) Seed Oil (and) Punica Granatum Flower Extract (and) Polyglyceryl-3 Diisostearate.
Oily extract of peony flowers: «Pivoine de France extrait huileux (amande douce raffinée)» by Greentech having the INCI name: Prunus Amygdalus dulcis (sweet almond) oil (and) Paeonia officinalis flower extract.

These extracts are used at a concentration of 0.25% by weight of raw material in the culture medium in the presence of 1% BSA (Bovine Serum Albumin) to solubilise the oily active ingredients.

1.2. Culture and Treatment of Normal Human Keratinocytes

Normal Human Keratinocytes (NHKs) obtained from an abdominoplasty were seeded at passage 4 in TPP150 flasks in supplemented Epilife medium. The NHKs were trypsinized to pre-confluence (0.05% Trypsin-EDTA, GIBCO-Invitrogen), and neutralized with a serum-containing medium. They were afterwards seeded at passage 5 in 96-well imaging plates (μ-plate 96 welt, ibiTreat, Ibidi) in a proportion 15 000 cells per well.

At confluence, the NHKs were treated with the plant extracts of the invention in complete Epilife medium containing 200 μM of CaCl₂), to promote cell differentiation (0.2 mL/well). Treatment was performed in triplicate for each condition, for 48 h (study of early differentiation markers) or for 5 days (study of late differentiation markers). For non-treated control conditions (NT), the medium was replaced by fresh medium+CaCl2).

1.3. Culture and Treatment of Normal Human Keratinocytes

Normal human fibroblasts (NHFs) obtained from an abdominoplasty were seeded in DMEM medium 1 g/L glucose supplemented with 10% foetal calf serum at passage P6 with a seeding density of 30 000 cells per well, in 96-well plates.

At confluence, the NHFs were treated with the plant extracts of the invention in DMEM medium 1 g/L glucose +10% FCS. Treatment was performed in triplicate for each condition, for 5 days. For control conditions NT (not treated) the medium was replaced by fresh medium.

1.4. Immunostaining

After these treatments, the cells were rinsed three times with PBS (PBS Tablets, Invitrogen GIBCO), and fixed with formalin (Formalin Solution 10% Neutral Buffered, Sigma) for 10 minutes.

After rinsing 3 times with PBS, the cell membranes were permeabilized with 0.1% PBS Triton solution (Triton X-100, Sigma), and again rinsed 3 times with PBS. The cells were coated with 1% PBS/BSA solution (Albumin, from bovine serum, Sigma), for 30 minutes, at ambient temperature.
The 1% PBS/BSA solution was then replaced by a primary antibody solution corresponding to each stained protein (see Table 1) diluted in 1% PBS/BSA. The plates were left to incubate overnight at 4° C.
The cells were rinsed three times with PBS and coated with a secondary antibody solution according to the primary antibody to be targeted (see Table 2) and with 1:500 DAPI (4′,6′-diamidino-2-phenylindole, dihydrochloride, Invitrogen Molecular Probes) diluted in 1% PBS/BSA. The plates were kept in the dark for one hour at room temperature.
The secondary antibody solution was aspirated and the cells rinsed twice with PBS and once with distilled water. PBS was deposited in each well in a proportion of 1 mL. The plates were kept in the dark at 4° C., until image acquisition.

TABLE 1
List of stained targets and conditions of use

| | | | Permeabil- | | |
|---|---|---|---|---|---|
| Target name | Supplier | Reference | ization | Dilution | Species |
| Aquaporin 3 | Abcam | Ab 215853 | + | 1:200 | Rabbit |
| Nidogen | R&D | AF2570 | − | 1:200 | Goat |
| | system | | | | |
| Procollagen | Abcam | Ab | + | 1:1000 | Rat |
| type 1 | | 64409 | | | |
| Fibronectin | Novotec | 24911 | − | 1:500 | Rabbit |

1.5. Image Acquisition and Analysis

The plates were scanned with ArrayScan XTi by Thermo Cellomics.

The images were analysed with Spot Detector image analysis software, a bio-application developed for the Arrayscan system.

This programme (pipeline) allows detection of red staining of the targeted protein, corresponding to the expression thereof. The surface of the measuring zone is delimited as being the entirety of the image surface.

The number of cells was determined by count of nuclei, via detection of blue staining.

1.6. Statistical Analysis

Statistical analysis of results was carried out by computing the mean, standard deviation and confidence interval (α=0.05).

The significance of the differences in these ratios observed between the controls and the treated cells was measured with Student's test. This test is said to be significant if the value obtained is less than 0.05.

### 2. In-Vitro Effects of the Plant Extracts of the Invention

The effect of the plant extracts of the invention and of the association thereof was tested on NHK and NHF cultures. The NHKs and NHFs were placed in culture and treated following the above-described protocols, to evaluate the gene expression of the proteins of interest.

2.1. Effect on Hydration

Aquaporins (AQPs) are a class of membrane proteins which form «pores» or channels permeable to water molecules in biological membranes. They allow the supply of water from the innermost layers towards the surface layers; AQP3 expressed in the lower part of the epidermis therefore plays a fundamental role in setting up trans-epidermal water flow. The advantage thereof in regulating skin hydration has been particularly investigated by the Applicant.

The results in FIG. 1 show a very strong, significant increase (+846%) in staining intensity of Aquaporin 3 when treated with the oily extract of pomegranate atone. The peony extract alone has no beneficial effect on the expression of this gene. However, the association of both extracts induces a significant increase (+932%) in the expression of Aquaporin 3, even higher than that obtained with the pomegranate extract atone.

These results therefore show that the association of the two extracts of the invention has beneficial effects on the hydration of keratin material, in particular the skin and/or lips.

2.2. Effect on the Dermal-Epidermal Junction

The dermal-epidermal junction or epidermal basement membrane is the acellular region separating the dermis from the epidermis. It provides mechanical support for adhesion of the epidermis to the dermis, controls exchanges and plays a fundamental role in skin re-epidermisation by acting, via its constituent glycoproteins, as support for adhesion and migration of keratinocytes. Nidogen (also called entactin), is a glycoprotein found at this basal lamina which allows the assembling of the constituents of this basal lamina.

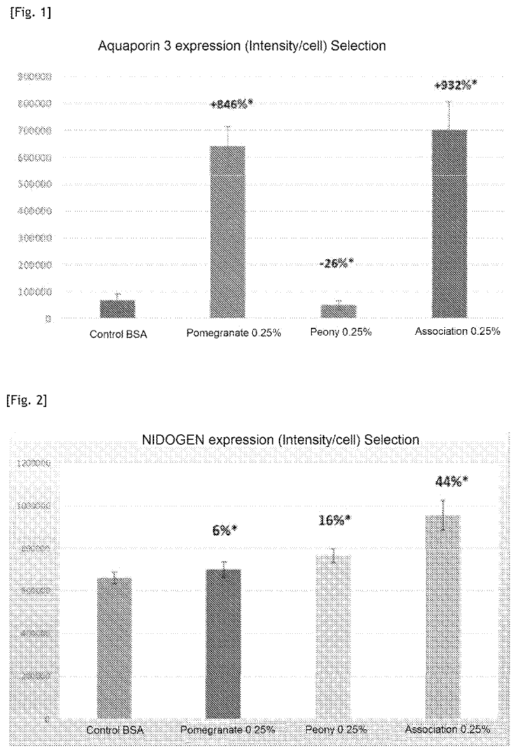

The results in FIG. 2 show that treatment with the oily extract of pomegranate atone has no effect on the expression of Nidogen. Solely the peony extract alone allows a significant increase (+16%) in the expression of this gene. Unexpectedly, the results also show that the association of these two extracts produces a synergic effect with a significant increase of 44% in the expression of Nidogen.

Unexpectedly, the results therefore show that the association of the two extracts of the invention exhibit synergic effects beneficial for maintaining the dermal-epidermal junction and hence the barrier function of the skin.

2.3. Effect on Firmness and Volume

Collagen 1 belongs to a family of so-called structural proteins and is present in fibril form in the dermis. This supporting protein is abundantly contained in the extracellular matrix (ECM), providing tissues with mechanical resistance to stretching. Procollagen is the soluble precursor of type 1 collagen. The depositing and assembling of collagen fibrils in the ECM is a complex process involving several proteins including the structural protein fibronectin.

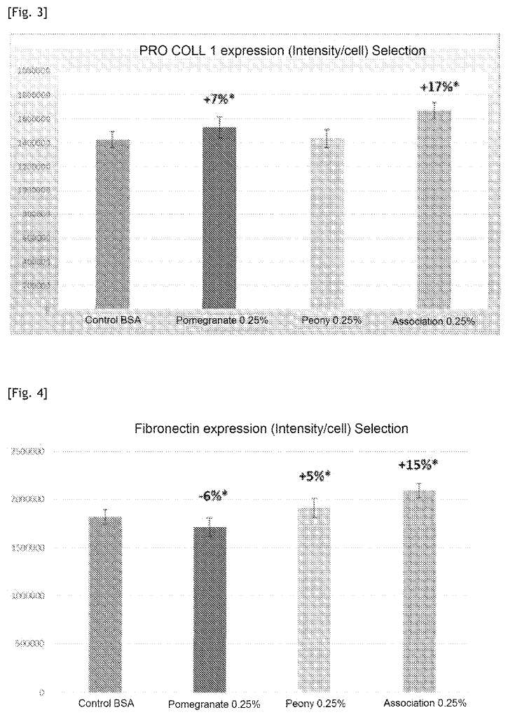

The results in FIG. 3 show that neither treatment with the oily extract of pomegranate alone nor treatment with the peony extract alone allow modulation of the expression of procollagen 1. On the other hand, and unexpectedly, the results show that the association of these two extracts produces a synergic effect by allowing a significant increase of 17% in the expression of this gene.

Additionally, the results in FIG. 4 show that treatment with the pomegranate extract alone leads to a significant decrease of 6% in the expression of fibronectin, whilst the peony extract alone significantly increases the expression of fibronectin by 5%. Surprisingly, the association of the two extracts produces a potentializing effect, by allowing a significant increase of 15% in the expression of fibronectin.

Therefore, surprisingly, the results show that the association of the two extracts of the invention exhibits synergic, even potentializing effects, and hence beneficial for maintaining of the skin extracellular matrix. This association of extracts therefore allows the preventing and/or reducing of toss of volume, loss of firmness, toss of elasticity and decline in skin thickness

### 3. Examples of Formulations

The cosmetic compositions illustrated below were prepared following conventional formulation methods in the field of cosmetics.

3.1. Composition in the Form of a Rich Cream

TABLE 2

| Ingredients | Weight % |
|---|---|
| Glycerine | 7.50 |
| Acrylates/C10-30 alkyl acrylate crosspolymer | 0.30 |
| Xanthan gum | 0.20 |
| Cetyl alcohol/glyceryl stearate/PEG-75/ceteth/steareth-2 | 5.25 |
| C10-C18 triglycerides | 5.00 |
| Hydrogenated coco-glycerides | 2.50 |
| Cetyl alcohol | 1.00 |
| Hydrogenated polyisobutene | 8.20 |
| Citric acid | 0.02 |
| Trisodium citrate | 0.14 |
| Oily extract of pomegranate* | 0.25 |
| Oily extract of peony* | 0.25 |
| Tocopheryl acetate | 0.20 |
| Fragrance, neutralizer, preserving agent | QS |
| Purified water | QS to 100% |

*such as described in Materials and Method

The composition comprises an association of the two oily extracts of the invention, in equal proportions (0.25% oily extract of pomegranate flowers and 0.25% oily extract of peony flowers).

Applied to the skin, in particular to the face, this composition provides hydrating and nourishing action and allows the preventing and/or reducing of toss of volume, loss of firmness, toss of elasticity and decline in skin thickness

3.2. Composition in the Form of a Lip Balm

TABLE 3

| Ingredients | Weight % |
|---|---|
| Silica | 10% |
| Nylon powder | 7% |
| Polyethylene wax | 17% |
| Glycerine | 14% |
| Oily extract of pomegranate* | 0.25% |
| Oily extract of peony* | 0.25% |
| Isopropyl myristate | QS to 100% |

*such as described in Materials and Method

The composition comprises an association of the two oily extracts of the invention, in equal proportions (0.25% oily extract of pomegranate flowers and 0.25% oily extract of peony flowers).

Applied to the lips, this balm imparts an immediate nourishing and hydrating effect, it prevents toss of volume, protects sore lips and provides the lip surface with comfort, emollience and hydration.

1. A cosmetic composition comprising, in a physiologically acceptable medium, an extract of pomegranate and an extract of peony, wherein said pomegranate extract is an oily extract of pomegranate flowers and wherein said peony extract is an oily extract of peony flowers.

2. The cosmetic composition according to claim 1, wherein said oily extract of pomegranate flowers is an extract of pomegranate flowers in jojoba oil.

3. The cosmetic composition according to claim 1, wherein said oily extract of peony flowers is an extract of peony flowers in sweet almond oil.

4. The cosmetic composition according to claim 1, wherein said cosmetic composition comprises:
a. an extract of pomegranate flowers in jojoba oil, in a content ranging from 0.0001 to 10% by weight of raw material relative to the total weight of said composition;
b. an extract of peony flowers in sweet almond oil, in a content ranging from 0.0001 to 10% by weight of raw material relative to the total weight of said composition;
c. optionally shea butter.

5. The cosmetic composition according to claim 1, wherein said composition is in the form of a topical composition for the lips and/or skin.

6. A cosmetic method for the care and/or makeup of keratin material, comprising the application to said keratin material of at least one layer of a cosmetic composition such as defined in claim 1.

7. The cosmetic method according to claim 6, wherein said method is intended to promote and/or stimulate the hydration and/or nourishment of keratin material, and/or to prevent and/or reduce loss of volume, loss of firmness, loss of elasticity, decline in skin thickness, and/or deterioration of the barrier function of the skin and/or lips.

8. A method for promoting and/or stimulating the hydration and/or nourishment of keratin material and/or preventing and/or reducing loss of volume, loss of firmness, loss of elasticity, decline in thickness of the skin, and/or deterioration of the barrier function of the skin comprising administering to a subject in need thereof an association of plant extracts according to claim 1.
